(19) United States
(12) Patent Application Publication
(10) Pub. No.: US 2007/0239179 A1
(43) Pub. Date: Oct. 11, 2007

(54) Compliant Gastroplasty: Devices And Methods

(75) Inventor: Mark S. Ortiz, Milford, OH (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: Ethicon Endo-Surgery, Inc., Cincinnati, OH (US)

(21) Appl. No.: 11/278,259

(22) Filed: Mar. 31, 2006

(51) Int. Cl. A61B 17/08; A61B 17/10

(52) U.S. Cl. 606/153; 606/139

(57) ABSTRACT

Devices and methods of forming a compliant gastroplasty are presented. In general, fasteners that can reversibly couple are inserted into gastric tissue to form a gastric restriction. When an expanding pressure in the stomach exceeds a certain limit, the coupled fasteners can decouple to accommodate and relieve the excess pressure. Upon subsequent stomach shrinkage, fasteners are brought back into proximity and recouple to reform the gastroplasty. In particular, magnetic fasteners can be inserted into gastric tissue by injecting a solidifying composition into the tissue to form the fasteners. Various devices and methods for forming the gastroplasty are discussed.

### FIELD OF THE INVENTION

The present application is directed to devices and methods for forming a gastroplasty, and in particular to such devices and methods that can effect a gastroplasty having a compliant nature.

### BACKGROUND OF THE INVENTION

Severe obesity is a major health risk that can decrease life expectancy and give rise to a number of other associated ailments including the onset of cardiovascular disease, hypertension, diabetes and severe arthritis. A number of surgical procedures can be performed to aid in the treatment of obesity. One example is a gastric restriction, also known as a gastroplasty, in which one or more fasteners are inserted into gastric tissue to hold the tissue in a folded configuration that reduces the effective volume of a patient's stomach.

Due to the chronic, fluctuating forces acting upon the gastric walls of a patient and the constant movement of the stomach, gastric restrictions often have a limited lifetime. For example, large chronic forces (i.e., pressures) can act in the stomach due to any number of circumstances such as super-physiological events. Since current fasteners form a non-compliant coupling between the walls of the stomach, pressures that exceed the ability of the fasteners to maintain gastric tissue coupling may be relieved by unintentional separation of fasteners from the gastric walls. In such an instance, the gastric restriction needs to be surgically reapplied, which is troublesome for both the surgeon and the patient.

Accordingly, a need exists for forming gastric restrictions that are more robust to the cyclical forces acting on a patient's stomach. Furthermore, a need for devices that can create such gastric restrictions also exists.

### SUMMARY OF THE INVENTION

An exemplary embodiment is directed to a system for deploying one or more fasteners in gastric tissue to create a gastroplasty. The gastric restriction can be formed by arranging fasteners in a selected pattern. The system includes an insertion element having an end effector that can be endoscopically deployable (e.g., by a trans-oral route). One or more tissue positioning structures can be positioned on the end effector, along with one or more tissue penetrating probes. The tissue positioning structure can be embodied as one or more suction ports that are effective for adhering tissue to the structure. A trough can also be included with the tissue penetrating structure, one or more suction ports being optionally coupled to one or more walls of the trough. A tissue penetrating probe can be selectively deployable through a portion of the insertion element. For example, a probe can be advanced out of, and/or retracted from, an end effector using a variety of mechanisms, such as engaging a set of gear teeth on the probe with a rotatable wheel. In another example, the probe can be advanced into the trough through an opening. The probe can be configured to penetrate tissue (e.g., using a distal penetrating tip), and can also include a tissue stop for limiting probe penetration through tissue. A flexible elongate body can be included to help orient the probe. The probe can also be configured to deliver a fastener forming composition through one or more lumens to yield a fastener. For example, the probe can include two or more lumens and a static mixing nozzle for delivering the fastener forming composition. Potential fastener forming compositions include solidifiable gel mixtures that can include magnetic particles that tend to align their magnetic dipoles. Multiple probes and multiple troughs can be utilized with the end effector to deliver the fasteners. For example, two tissue penetrating probes can be located on opposing walls of a trough to penetrate adjacent tissue, or separate troughs can be configured to adhere gastric tissue on the anterior side and on the posterior side of the stomach.

Another exemplary embodiment is directed to a gastric restriction fastener system that includes multiple magnetic fasteners. Fasteners can be shaped with a narrow intermediate portion configured to extend through gastric tissue, and widened portions on the ends for placement adjacent to a gastric tissue surface. Fasteners can be adapted to be embedded in gastric tissue, and oriented in a desired pattern to effect a gastroplasty of a patient's stomach. As well, each of the fasteners can be configured to magnetically adhere to one or more other fasteners. Coupled fasteners can also reversibly decouple in response to a separation pressure in excess of a predetermined threshold value (e.g., a value between about 1 pound per square inch and about 3 pounds per square inch).

Another exemplary embodiment is directed to a method of creating a compliant gastroplasty. Multiple fasteners, which each reversibly couple to one or more other fasteners, can be inserted into a gastric wall in a selected pattern to form a restricted volume within a stomach. For example, one or more lines of fasteners can be inserted, and such lines can be located on the anterior and posterior walls of the stomach to effect the gastric restriction. Types of fasteners include magnetic fasteners that tend to magnetically couple to another magnetic fastener. Fasteners can be formed from a solidifying composition that is inserted into the gastric wall. When a magnetic fastener is formed using a solidifying composition, the composition can include a dispersion of magnetic particulates. Fasteners can be configured to tend to decouple when the restricted volume of the stomach is subjected to a separation pressure in excess of a predetermined value, such as about 1 pound per square inch. The method can be performed endoscopically (e.g., trans-orally) to utilize a minimally-invasive surgical technique.

### BRIEF DESCRIPTION OF THE DRAWINGS

The invention will be more fully understood from the following detailed description taken in conjunction with the accompanying drawings, in which:

FIG. 1 presents an anterior view of a stomach having a plurality of fasteners attached thereto consistent with an embodiment of the invention;

FIG. 2 presents a cross-sectional view of a stomach having reversibly coupled fasteners attached to an anterior wall and a posterior wall of the stomach;

FIG. 3A presents an anterior view of a stomach with a cutaway portion showing an end effector of a fastener deployment system within the stomach cavity;

FIG. 3B a close up view of the cutaway portion of the stomach shown in FIG. 3A;

FIG. 3C shows a perspective view of an end effector of a fastener deployment system having a layer of tissue oriented within a trough of the end effector;

FIG. 4 shows a perspective view of a tissue layer with a tissue penetrating probe piercing the tissue layer;

FIG. 5 shows a perspective view of a tissue penetrating probe; and

FIG. 6 shows a fastener embedded in a gastric wall.

### DETAILED DESCRIPTION OF THE INVENTION

Certain exemplary embodiments will now be described to provide an overall understanding of the principles, structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with features of other embodiments. Such modifications and variations are intended to be included within the scope of the present invention.

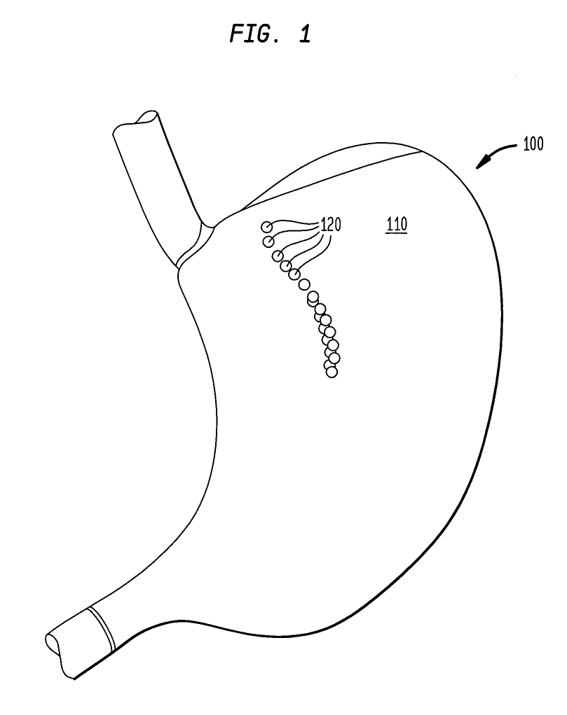

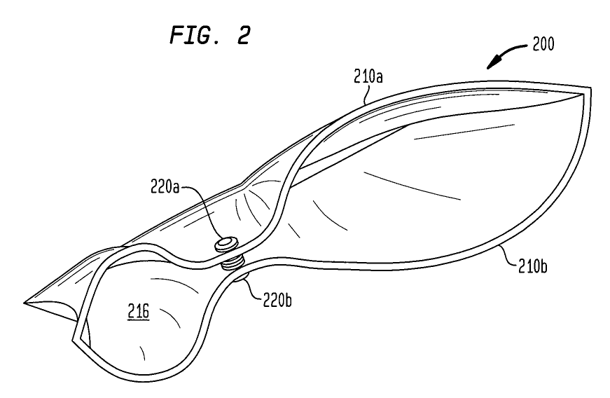

FIG. 1 presents an illustration of a gastric restriction fastener system coupled to an anterior side of a stomach 100, consistent with an exemplary embodiment. The system includes a plurality of fasteners that are embedded into gastric tissue and oriented in a desired pattern to effect a gastroplasty. For example, a set of fasteners 120 can be embedded into the anterior wall 110 of the stomach, as shown in FIG. 1, with a corresponding set of fasteners also embedded into the posterior wall (not shown). As depicted in the cross-sectional view of a stomach 200 in FIG. 2, the volume of the stomach 200 can be effectively restricted to a small volume 216 by the coupling of fasteners 220a, 220b that are each embedded in an opposed gastric wall 210a, 210b, respectively. In general, each of the fasteners 220a can be adapted to reversibly couple to at least one other fastener 220b. For example, fasteners can be in contact with one another during coupling, while sufficient tensile forces can cause the fasteners to separate. Upon sufficient relief from the tensile forces, the fasteners can recouple to contact each other again. Accordingly, fasteners 220a, 220b can be adapted to reversibly decouple in response to a separation pressure within the stomach 200 in excess of some predetermined value.

In use, reversibly coupled fasteners are typically coupled to form a gastric restriction. If some event causes the pressure in the stomach to exceed a predetermined pressure value, the reversibly coupled fasteners can decouple to effectively increase the volume of the stomach, and thereby relieve the excessive pressure without causing the fasteners to be torn or removed from the gastric wall. When the pressure in the stomach returns to a nominal level, shrinkage of the stomach can cause the anterior and posterior gastric walls to approach one another. Fasteners that are brought back into proximity to one another by the contracting stomach walls can recouple, thereby reforming the gastric restriction. In general, if a set of fasteners are within the anterior wall of the stomach (as shown in FIG. 1 for example), with a corresponding set of fasteners embedded within the corresponding posterior wall, each fastener in the anterior wall need not attach to an exact corresponding fastener in the posterior wall. Indeed, after decoupling and recoupling to reform a gastric restriction, some fasteners may not be coupled to another fastener, or multiple fasteners may be coupled to one fastener. The reversible nature of fastener coupling can allow a gastric restriction to form, break apart, and reform, i.e., the gastric restriction can be self-correcting. As such, the gastric restriction fastener system provides a compliant gastroplasty that can withstand super-physiological events.

Fasteners utilized with a gastric restriction fastener system can be configured in a variety of shapes and sizes to reversibly couple and decouple in a manner consistent with a compliant gastroplasty. For example, the fastener can be configured to penetrate gastric tissue, with a narrow intermediate portion 352 extending through the tissue and wider portions 351, 353 located on each end of the intermediate portion 352 that are adjacent to a tissue surface 420 as exemplified by the fastener 350 depicted in FIG. 6. As mentioned earlier, fasteners can be arranged in a desired pattern to form the gastric restriction. The restriction can proceed along a path extending from near the cardiac orifice to the pylorus, or some intermediate path as depicted by the restriction shown in FIG. 1. Any number of fasteners can be used to create the reversible gastric restriction. For example, pairs of coupled fasteners can be located about 2 millimeters to about 8 millimeters apart, or preferably about 4 millimeters to about 5 millimeters apart. As well, one or more linear or non-linear rows of fasteners can be used to form the restriction.

Fasteners can also be constructed from a variety of materials. In one embodiment, each fastener can be embodied as a magnetic fastener that can magnetically adhere to one or more other fasteners. As such, the magnetic fastener can also be configured to reversibly couple and decouple. In particular, a magnetic fastener can be formed from a fastener forming composition (e.g., a gel composition) that solidifies into a final fastener shape. The fastener forming composition can include magnetic particulates that tend to align their magnetic dipoles. One example of a fastener forming composition is a gel composition of polymer solution including polyvinyl alcohol combined with nanosized iron oxide particles (e.g., γ-Fe₂O₃,˜7 nm), as described by Chattergee et al. (BioMagnetic Research and Technology 2004, 2:2), the entire contents of which are hereby incorporated herein by reference. Upon mixing, the composition quickly forms a gel that dries and solidifies. As such, the gel can quickly set into a desired shape, such as that previously described. Those skilled in the art will readily appreciate that a number of other types of compositions or other types of magnetic fasteners (e.g., preformed fasteners) or non-magnetic fasteners can be utilized to form a compliant gastroplasty. These various types of fasteners are all included within the scope of the present application.

As previously mentioned, reversible decoupling of fasteners can occur when the pressure within the stomach exceeds a predetermined value. For example the predetermined value can be greater than about 1 pound per square inch, or in the range of about 1 pound per square inch to about 3 pounds per square inch, or the predetermined value can be about 2 pounds per square inch. When magnetic fasteners are utilized, the strength of the magnetic attraction between fasteners can be regulated to obtain the desired predetermined value at which a gastric restriction would be released to relieve internal stomach pressure.

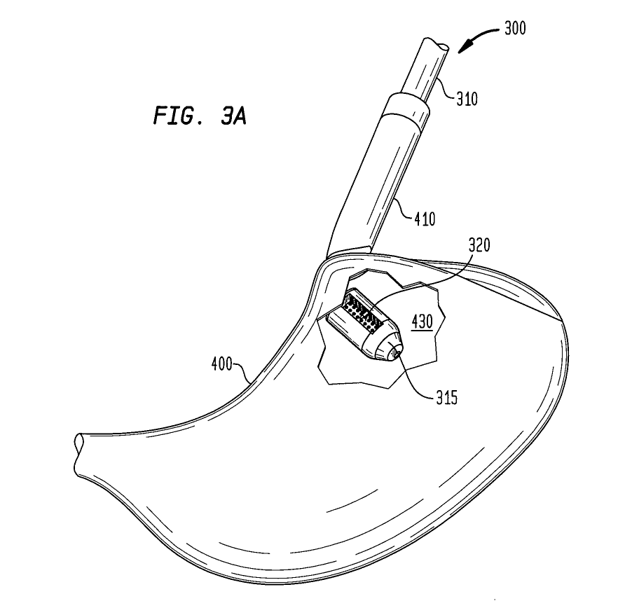

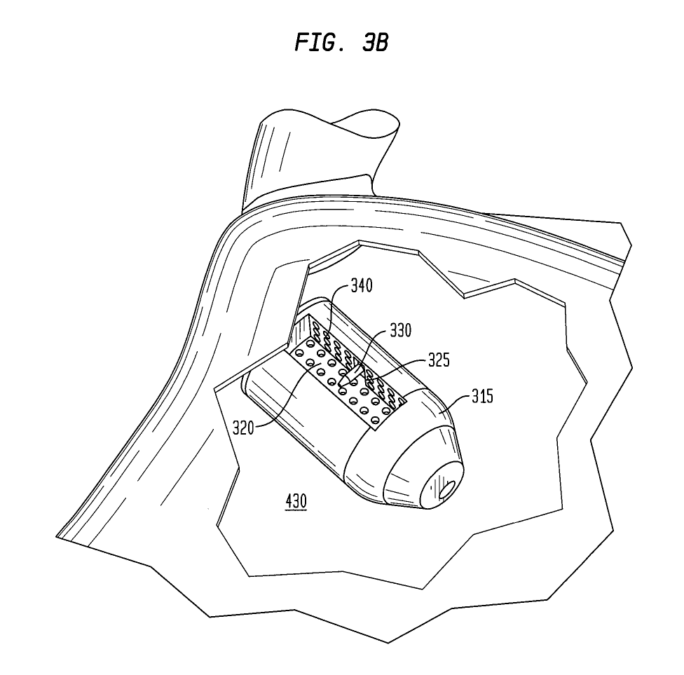

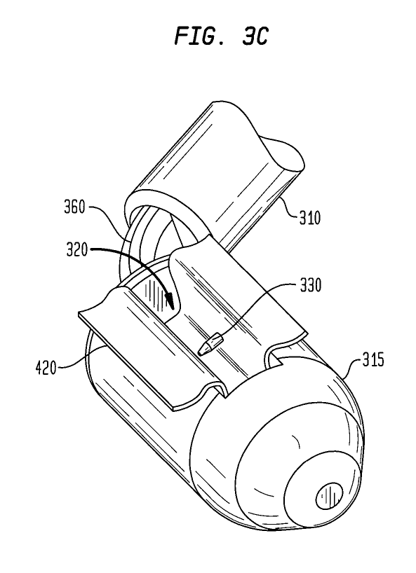

Another embodiment is directed to a system for deploying fasteners in gastric tissue to effect a gastroplasty. Such a system is generally represented by the device 300 depicted in FIGS. 3A-3C. The device 300 can include an endoscopically deployable insertion element having an end effector 315. The end effector 315 can be coupled to a shaft 310 of the insertion element by a coupling 360 that can be configured to advance and/or angle the end effector 315. The end effector can include one or more tissue positioning structures, and can also be associated with one or more tissue penetrating probes that can be selectively deployable through a portion of the insertion element. For example, as depicted in FIG. 3B, the probe 330 can be deployed through a slot 325 (or other opening) and into the trough 320 of an end effector 315. A probe can also include one or more lumens for delivering a fastener forming composition. One skilled in the art will appreciate that the device 300, including shaft 310 and end effector 315, is of a type of construction that is suitable for endoscopic delivery, such as trans-oral delivery. For example, shaft 310 can be an elongate member that is of sufficient flexibility, and/or selectively flexible, to traverse a tortuous path within a lumen of the body.

In general, tissue positioning structures can orient and/or position tissue (e.g., the gastric wall) to facilitate insertion of a fastener element. For the exemplary embodiment shown in FIGS. 3A-3C, the end effector 315 includes a trough 320 having one or more suction ports 340 disposed in the inner wall. A suction port can be configured to adhere tissue thereto, such as depicted in FIG. 3C where the suction ports 340 draw the gastric tissue 420 into the trough 320. A trough configuration can be advantageous by providing isolation for a tissue penetrating probe that pierces the gastric tissue, and hindering potential collateral damage that can be associated with tissue piercing by the probe. As depicted by the anterior cutaway view of the stomach shown in FIG. 3A, the end effector 315 can be configured with a trough 320 that can be positioned to adhere gastric tissue 400 from the anterior side of the stomach. The end effector 315 can also include an additional trough (not shown) opposite the illustrated trough 320 for adhering gastric tissue from the posterior side of the stomach. In such a configuration, an end effector can apply a fastener to each side of the stomach (as shown in FIG. 2) for forming the gastric restriction. Though FIGS. 3A-3C show an exemplary embodiment of an end effector with a tissue positioning structure, those skilled in the art will readily appreciate that a variety of other tissue positioning structure configurations can be effectively utilized with a fastener deployment system. For example, the use of a trough is not required as part of tissue positioning structure. One or more suction ports can be utilized without a trough for adhering tissue. Furthermore, other types of tissue positioning structures besides a suction port can be used to orient tissue in a desired configuration for penetration by a tissue penetrating probe (e.g., tissue graspers, clamps, and other tissue manipulating devices). All of these variations are within the scope of the present application.

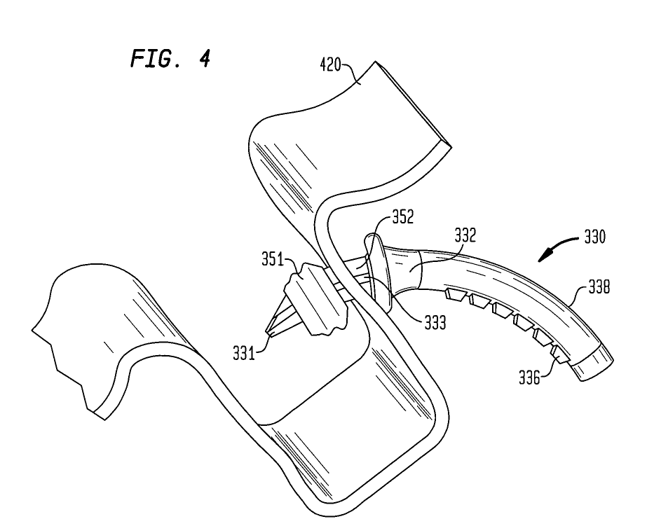

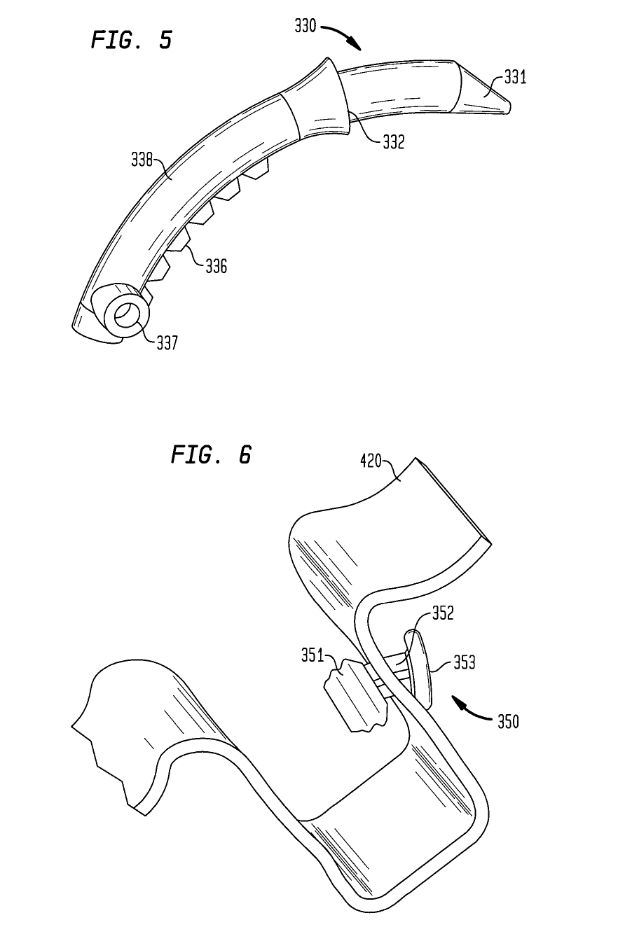

Tissue penetrating probes used with an insertion element can be configured in variety of manners to allow tissue penetration and probe manipulation (e.g., advancement out of, or retraction into, an end effector). An exemplary probe 330 is depicted in FIG. 5. A probe 330 can include a distal penetrating tip 331 for tissue penetration. The probe can also include a tissue stop configured to limit penetration of the probe into the tissue, such as the inverted flare structure 332 shown in FIGS. 4 and 5. A flexible elongate body 338 can be used to aid orientation and manipulation of the probe 330. A plurality of gear teeth 336 can be distributed along a portion of the probe 330 and configured to engage a rotatable gear wheel (not shown), which can be located within the insertion element. By rotating the gear wheel in a desired direction, the probe can be advanced or retracted. Those skilled in the art will readily appreciate that probes can be configured in a variety of other configurations beyond what is described herein, and can be utilized with tissue positioning structures in a various arrangements. For example, two tissue penetrating probes can be deployed through opposing inner walls of a trough and into adjacent tissue to form fasteners therein, either serially or simultaneously. Indeed, such arrangements, among others, can deliver a plurality of fasteners in a selected pattern arrangement to aid in forming a gastric restriction.

As previously discussed, a probe 330 can include a lumen 333 (shown in FIG. 4) for delivering a fastener forming composition as described herein (e.g., a solidifiable gel mixture), the composition entering the lumen through a port 337 as depicted in FIG. 5. Since some fastener forming compositions solidify quickly under particular conditions, it can be advantageous to configure a probe to compartmentalize stable components of the composition and combine the components prior to distributing the composition to yield a fastener. For example, a probe can contain two or more lumens and a static-mixing nozzle, with the plurality of lumens holding separate components of the fastener forming composition. Upon moving the components of each lumen into the nozzle, the components are mixed to form the fastener forming composition. The components, or the entire premixed fastener forming composition, can be stored within the probe 330 (e.g., within the one or more lumens or in one or more reservoirs in the probe). Alternatively, the components or entire composition can be stored elsewhere within the fastener deployment system. For example, the materials can be stored in one or more separate reservoirs, and can be subsequently delivered to the probe. Such reservoirs can be stored within a portion of the insertion element, or can be completely separated from the insertion element, but placed in fluid communication with the insertion element by flexible tubing or other portal.

In use, with reference to FIGS. 3A-3C, a fastener deployment system can be configured to be delivered trans-orally, through the esophagus 410 and into the cavity 430 of the stomach, as shown in the cutaway view of FIGS. 3A and 3B. Suction can be applied through the ports 340 to adhere gastric tissue 420 into the trough 320 of the end effector 315 depicted in FIG. 3C. A tissue penetrating probe 330 can be advanced through tissue as shown in FIG. 3C and the close-up perspective view of FIG. 4. The fastener forming composition can then be delivered through the probe 330 to form a fastener, such as the fastener 350 shown in FIG. 6. In general, the probe can be retracted or advanced as the fastener is formed, with the rate of composition release being controlled to aid in fastener shape formation. Upon composition solidification, a fastener is formed into the gastric wall. This process can repeated multiple times to form multiple fasteners. Accordingly, the composition is oriented to create a fastener with poles oriented to induce attraction with another fastener. Typically, magnetic particulates in a dispersion can orient themselves. However, a magnetic pole can be induced in the end effector, or other section of the insertion element, to cause fasteners in the posterior wall to attract fasteners in the anterior wall. Alternatively, if the end effector, or insertion element, is slightly magnetic, particles can orient in response to the magnetic field. After fastener formation, the fastener can be separated from the end effector using light insufflation, or other separation techniques. Upon formation of fasteners in a desired pattern, one or more fasteners can couple, for example as shown in FIG. 2, to form a gastric restriction. Coupling can be promoted by bringing the opposed fasteners into proximity, such as by the application of suction within the stomach cavity.

Another exemplary embodiment is directed to a method of creating a compliant gastroplasty. The method can be performed endoscopically, such as in a trans-oral manner, with a plurality of fasteners being inserted into a gastric wall. The fasteners can be arranged in a predetermined pattern, such as in one or more lines extending in a manner to form a gastric restriction (e.g., one line in the anterior wall and a corresponding line in the posterior wall). One or more fasteners can be adapted to reversibly couple to at least one other fastener to form a restricted volume within a stomach. For example, the fasteners can be configured to decouple when the restricted volume is subjected to an expanding pressure in the stomach greater than about 1 pound per square inch. The fasteners can be magnetic, having a tendency to magnetically couple to another fastener. Such magnetic fasteners can be formed using a fastener forming composition in a dumbbell-like shape, as described herein, or can be preformed from a composition, or any other material, before being inserted into the gastric wall. It is clear to those skilled in the art that the exemplary method can be performed using any of the fasteners, gastric restriction fastener systems, and/or fastener deployment systems described herein. However, the method certainly does not require the use of any of the aforementioned devices or systems. For example, the method can be practiced by utilizing magnetic fasteners that are preformed (either with or without a fastener forming composition), and inserted into the gastric wall using existing fastener applying devices. As well, non-magnetic fasteners and more traditional surgical routes can also be utilized consistently with the method. All these variations and others are within the scope of the present application.

In another aspect, fastener deployment systems, including portions thereof, can be designed to be disposed of after a single use, or they can be designed to be used multiple times. In either case, however, the system can be reconditioned for reuse after at least one use. Reconditioning can include any combination of the steps of disassembly of the system, followed by cleaning or replacement of particular pieces, and subsequent reassembly. By way of example, the fastener deployment system shown in FIGS. 3A-3C and 5 can be reconditioned after the system has been used in a medical procedure. The device can be disassembled, and any number of the particular pieces (e.g., shaft 310, the end effector 315, the tissue penetrating probe 330 including any portions of the probe, etc.) can be selectively replaced or removed in any combination. For instance, the end effector can be replaced by a new end effector, while the remaining pieces of the insertion element are sterilized for reuse. Replacement of pieces can also include replacement of portions of particular elements, such as the replacement of a distal tip on a tissue penetrating probe. Upon cleaning and/or replacement of particular parts, the system can be reassembled for subsequent use either at a reconditioning facility, or by a surgical team immediately prior to a surgical procedure. Those skilled in the art will appreciate that reconditioning of a fastener deployment system can utilize a variety of techniques for disassembly, cleaning/replacement, and reassembly. Use of such techniques, and the resulting reconditioned system, are all within the scope of the present application.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present invention. As well, one skilled in the art will appreciate further features and advantages of the invention based on the above-described embodiments. Accordingly, the invention is not to be limited by what has been particularly shown and described, except as indicated by the appended claims.

What is claimed is:

1. A system for deploying fasteners in gastric tissue to effect a gastroplasty, comprising:
an endoscopically deployable insertion element having an end effector with at least one tissue positioning structure formed thereon; and
at least one tissue penetrating probe associated with the end effector and selectively deployable through a portion of the insertion element, the at least one probe configured to penetrate tissue and deliver a fastener forming composition through a lumen of the at least one probe to yield a fastener that extends through opposed walls of the gastric tissue.

2. The system of claim 1, wherein the at least one probe includes at least two lumens and a static-mixing nozzle for delivering the fastener forming composition.

3. The system of claim 1, wherein the at least one probe is configured to deliver a magnetic fastener forming composition.

4. The system of claim 3, wherein the magnetic fastener forming composition comprises a solidifiable gel mixture.

5. The system of claim 4, wherein the solidifiable gel mixture comprises magnetic particles tending to have aligned magnetic dipoles.

6. The system of claim 1, wherein the at least one probe includes a distal penetrating tip and a tissue stop located proximal to the tip for limiting penetration of the at least one probe through the tissue.

7. The system of claim 1, wherein the at least one probe is configured to be advanced out of the end effector and retracted into the end effector.

8. The system of claim 1, wherein the at least one probe includes a flexible elongate body.

9. The system of claim 1, wherein a plurality of gear teeth are distributed along a section of the at least one probe for engaging a rotatable gear wheel in the insertion element that advances and retracts the at least one probe.

10. The system of claim 1, wherein the at least one tissue positioning structure includes at least one suction port effective to adhere tissue thereto.

11. The system of claim 10, wherein the at least one tissue positioning structure includes at least one trough for receiving tissue, the at least one suction port coupled to an inner wall of the at least one trough.

12. The system of claim 11, wherein the at least one probe includes two tissue penetrating probes configured to be deployed through opposing inner walls of the at least one trough and into adjacent tissue.

13. The system of claim 11, wherein the at least one tissue positioning structure includes at least two troughs each having at least one suction port, one trough configured to adhere gastric tissue from an anterior side of the stomach and another trough configured to adhere gastric tissue from a posterior side of the stomach.

14. The system of claim 11, wherein the at least one probe is configured to advance into the trough through an opening in the trough.

15. The system of claim 1, wherein the device is configured to deliver a plurality of fasteners in a selected pattern.

16. The system of claim 1, wherein the insertion element is configured to be delivered trans-orally.

17. A gastric restriction fastener system, comprising:
a plurality of magnetic fasteners, each adapted to be embedded in gastric tissue and oriented in a desired pattern to effect a gastroplasty of a stomach in a patient, each of the fasteners being configured to magnetically adhere to at least one other fastener to form a gastric restriction and to reversibly decouple in response to a separation pressure in excess of a predetermined threshold value.

18. The gastric restriction of claim 17, wherein the predetermined value is in a range of about 1 pound per square inch to about 3 pounds per square inch.

19. The gastric restriction of claim 17, wherein at least one of the magnetic fasteners includes a narrow intermediate portion extending through gastric tissue and widened portions on each end of the intermediate portion adjacent to a side of the gastric tissue.

20. A method of creating a compliant gastroplasty, comprising:
inserting a plurality of fasteners to a gastric wall in a selected pattern, each fastener adapted to reversibly couple to at least one other fastener to form a restricted volume within a stomach.

21. The method of claim 20, wherein the selected pattern comprises at least one line of fasteners.

22. The method of claim 20, wherein the step of inserting the plurality of fasteners includes inserting a plurality of magnetic fasteners, each fastener tending to magnetically couple to at least one other magnetic fastener.

23. The method of claim 22, wherein the plurality of magnetic fasteners each comprise a composition including a dispersion of magnetic particulates.

24. The method of claim 20, wherein the step of inserting the plurality of fasteners includes inserting a solidifying composition into the gastric wall to form the plurality of fasteners.

25. The method of claim 24, wherein at least one of the fasteners includes a narrow intermediate portion extending through gastric tissue and widened portions on each end of the intermediate portion adjacent to a side of the gastric wall.

26. The method of claim 20, wherein the step of inserting the plurality of fasteners includes inserting the plurality of fasteners such that at least one fastener is attached to the anterior gastric wall and at least one fastener is attached to the posterior gastric wall.

27. The method of claim 20, wherein the step of inserting the plurality of fasteners is performed trans-orally.

28. The method of claim 20, wherein the plurality of fasteners tend to decouple when the restricted volume of the stomach is subjected to an expanding pressure greater than a predetermined separation pressure.
